(19) United States
(12) Patent Application Publication
(10) Pub. No.: US 2004/0013705 A1
(43) Pub. Date: Jan. 22, 2004

(54) Processed meat products capable of potentiating motor endurance

(76) Inventors: Itaru Watanabe, Shizuoka (JP); Yasuyuki Oishi, Shizuoka (JP); Hironori Tomi, Shiga (JP)

Correspondence Address: GREENBERG TRAURIG, LLP, 885 3RD AVENUE, NEW YORK, NY, 10022, US

(21) Appl. No.: 10/381,539

(22) PCT Filed: Sep. 27, 2001
(86) PCT No.: PCT/JP01/08424

(30) Foreign Application Priority Data
Sep. 28, 2000 (JP) 2000-295934
Jul. 3, 2001 (JP) 2001-201553

(51) Int. Cl. A61K 35/78; A61K 47/00

(52) U.S. Cl. 424/439; 424/777

(57) ABSTRACT

An object of this invention is to provide new processed meat foods that can fortify the physical endurance significantly or promote the combustion of body fats.

This invention relates to a processed meat food having mixed therein a Garcinia rind extract containing HCA's. In addition, this invention relates to a use of a Garcinia rind extract for the production of processed meat foods for physical endurance fortification or processed meat foods for body fat combustion promotion, which have mixed therein the Garcinia rind extract containing HCA's, or relates to a method of fortifying the physical endurance of a human being or other animal or promoting the body fat combustion by a human being or other animal, wherein a processed meat food, having HCA's mixed therein, is taken in by the human being or other animal.

### TECHNICAL FIELD

This invention relates to processed meat foods that can fortify physical endurance and processed meat foods that can promote body fat combustion. To be more specific, this invention relates to processed meat foods having hydroxycitric acid (referred to as “HCA” hereinafter) and its lactone form or a salt of either (−)-hydroxycitric acid and its lactone form (referred to collectively as “HCA's” hereinafter) mixed therein.

Here, “physical endurance” refers to the ability to maintain a fixed state of exercise. “Body fat combustion promotion” refers to the promotion of combustion of fats that exist in a body to enable the amount of fats in the body to be reduced.

### BACKGROUND ART

HCA's are well known to be contained in the rinds of Garcinia. Since HCA's can restrict the increase of body fat amount, Garcinia rind extracts have actually become widely popular as health foods for dieting. It has also become recently known that HCA's can fortify physical endurance (e.g. PCT WO98/35664).

### SUMMARY OF THE INVENTION

An object of this invention is to provide new processed meat foods that can fortify the physical endurance significantly or promote the combustion of body fats.

As a result of diligent examination, the present inventors have found that surprisingly, processed meat foods having HCA's mixed therein can fortify the physical endurance more significantly and promote the combustion of body fats in comparison to the case where HCA's are taken in solitarily, and have thus come to complete the present invention.

This invention thus provides a processed meat food having HCA's mixed therein. This invention also provides a processed meat food having mixed therein a Garcinia rind extract containing HCA's.

Other modes of this invention include the following.

(1) The use of HCA's for the production of processed meat foods for physical endurance fortification or processed meat foods for body fat combustion promotion, which have HCA's mixed therein.

(2) The use of a Garcinia rind extract for the production of processed meat foods for physical endurance fortification or processed meat foods for body fat combustion promotion, which have mixed therein the Garcinia rind extract containing HCA's.

(3) A method of fortifying the physical endurance of a human being or other animal or promoting the body fat combustion by a human being or other animal, wherein a processed meat food, having HCA's mixed therein, is taken in by the human being or other animal.

(4) A method of fortifying the physical endurance of a human being or other animal or promoting the body fat combustion by a human being or other animal, wherein a processed meat food, having mixed therein a Garcinia rind extract containing HCA's, is taken in by the human being or other animal.

Examples of salts of HCA include alkali metal salts, such as sodium salts, potassium salts, etc., alkali earth metal salts, such as magnesium salts, calcium salts, barium salts, etc., ammonium salts, ethylene diamine salts, etc. Among the above, sodium salts, potassium salts, and calcium salts are preferable.

Though HCA's can be produced by chemical synthesis (for example, Z. Physiol. Chem., 269, 33 (1941)), HCA's are generally obtained by extraction by a known method (for example, Phytochemistry, 4, 619-625 (1965)) from the rind of Garcinia that contains HCA's. For example, an extract containing HCA's can be obtained from Garcinia rind containing HCA's by extraction by a conventional method, such as, extraction in water or alcohol. The extract may be concentrated to an arbitrary concentration of HCA's by a conventional method and arbitrary salts of HCA, etc. can be formed by a conventional method during the extraction process or after extraction. Furthermore, the method indicated in U.S. Pat. No. 5,536,516 may be used to obtain an extract with a high HCA content and with which the amounts of components (for example, sugars) besides the HCA's are reduced. HCA and the lactone form of its salt may be prepared for example in accordance to the method of R. P. Singh, and others (Biol. Memoirs, 21, 27-33 (1995)). Furthermore, the extract may be made into a powder by a conventional method, such as the spray drying method, spray granulation method, etc., and using a suitable excipient (for example, dextrin, gum arabic, processed starch, etc.)

Examples of Garcinia that contain HCA's include goraka -(Garcinia cambogia Desr.), Indian mangosteen (Garcinia indica. Choisy), and Asam gelugor (Garcinia atroviridis Griff.).

The above-mentioned extract, etc. are known, and such known Garcinia extracts can be used as they are in this invention. For example, Garcinia Extract S (trade name; made by Nippon Shinyaku Co., Ltd.), Garcinia Powder S (trade name;, made by Nippon Shinyaku Co., Ltd.), Garcinia Powder J (trade name;, made by Nippon Shinyaku Co., Ltd.), Citrin (trade name; made by Sabinsa Corp. (USA)), and Citrimax (trade name; made by InterHealth Corp. (USA)) may be used as they are in this invention.

With this invention, “processed meat food” refers not to meat itself but refers to semi-products that contain meat, meat products, and meat-containing dishes (including semi-products. Here, “meat” refers to meats in the wide sense and includes not only the edible meats of fowls and mammals but also includes fish meat and whale meat.

Examples of semi-products that contain meat include raw hamburgers, raw Vienna sausages, raw seasoned meats, raw roast beefs, raw pork cutlets, raw chicken cutlets, and raw beef cutlets. Examples of meat products include meat hams, such as roast ham, boneless ham, pressed ham, chopped ham, lax ham, raw ham, etc., meat sausages, such as Vienna sausage, frankfurter, bologna sausage, loaf, dry sausage, salami sausage, etc., meat bacons, such as bacon, roast bacon, shoulder bacon, etc., meat corned beefs, such as corned beef, pressed corned beef, etc., roasted meat, such as roast pork, roasted pig, roast beef, etc., dried meats, such as beef jerky, pork jerky, dried meat, etc., hamburgers (including fish meat hamburgers), meat balls (including fish meat balls), minced meat cutlets, pork cutlets, chicken cutlets, beef cutlets, nuggets, and cooked seasoned meats. Examples of meat-containing dishes include raw shao-mai, raw jiao-zi, raw croquettes, raw spring rolls, hamburgers (including fish meat hamburgers), shao-mai, jiao-zi, croquettes, and spring rolls. Other examples of processed meat foods relevant to this invention include kamaboko (boiled fish paste), chikuwa (fish paste), rolled omelets, tsumire (dumplings for soup), hanpen (fish cake), naruto (fish cake), and satsuma-age (fried fish balls).

With this invention, HCA's are suitably contained at an amount in the range of 1 mg to 30 g per 100 g of processed meat food. HCA's are preferably contained at an amount in the range of 10 mg to 10 g per 10 g of processed meat food and more preferably at an amount in the range of 50 mg to 6 g per 100 g of processed meat food. The effects of this invention may be exhibited outside these ranges and these ranges do not limit the scope of this invention.

With this invention, “mixing” refers to the mixing of a processed meat raw material, especially, meat with HCA's or with an extract of Garcinia rind that contains HCA's.

Besides performing the mixing in, for example, of a prescribed amount of HCA's or a Garcinia extract containing HCA's in any of the production processes of a processed meat food, a processed meat food by this invention may be produced by the conventional methods.

As a specific example of the timing of mixing and method of mixing HCA's or a Garcinia extract containing HCA's in a processed meat food, in the case of hams or bacons, a so-called pickling solution, having HCA's or a Garcinia extract containing HCA's blended therein, may be loaded into the corresponding meat, or the HCA's or the Garcinia extract containing HCA's may be mixed in the process of making salt-pickled meat by pickling meat in a cover pickling solution, etc. or by rubbing in salts by the dry pickling method. In the case of sausages, HCA's or a Garcinia extract containing HCA's may be mixed by addition in the process of pickling and blending the corresponding meat, which has been chopped, with water and salt or other additive. In the case of hamburgers and meat balls, HCA's or a Garcinia extract containing HCA's may be mixed by blending and mixing in the process of mixing the corresponding meat, which has been chopped, with other blended components in a mixer.

A processed meat food by this invention may be taken in a normal manner for food once to few times a day, and can be taken in at the same time as the normal diet.

A processed meat food by this invention is useful for significantly fortifying the physical endurance of a human being or other animal. A processed meat food by this invention is also useful in promoting the combustion of body fats by a human being or other animal and can reduce the amount of body fats.

### BEST MODE FOR CARRYING OUT THE INVENTION

This invention shall now be described in further detail by description of examples and test examples. Needless to say, this invention is not limited to the examples described below.

### EXAMPLE 1

### Hams Containing HCA's

Besides loading 15 kg of a pickling solution of the recipe shown in Table 1 or 2 into 100 kg of pork, which had been removed of bones and extraneous matter, adjusted in the amount and thickness of fat tissue, and cut to portions of suitable size, to prepare pickled meat, two types of processed meat food by this invention (hams containing HCA's (0.5 weight % HCA is contained in the case of the recipe of Table 1 and 1.5 weight % HCA is contained in the case of the recipe of Table 2)) were produced through conventional processes.

TABLE 1

| | Pickling | |
|---|---|---|
| | solution | Pickled meat |
| | composition % | composition % |
| Garcinia Powder J | 6.4 | 0.83 |
| (trade name) (contains | | |
| 60% HCA) | | |
| Common salt | 14 | 1.83 |
| Phosphate | 2 | 0.26 |
| Nitrite | 0.1 | 0.01 |
| Ascorbate | 0.4 | 0.05 |
| Sugar | 5 | 0.65 |
| Sodium glutamate | 1 | 0.13 |
| Water | 71.1 | |
| Total | 100 | |

TABLE 2

| | Pickling | |
|---|---|---|
| | solution | Pickled meat |
| | composition % | composition % |
| Garcinia Powder J | 19.2 | 2.49 |
| (trade name) (contains | | |
| 60% HCA) | | |
| Common salt | 14 | 1.83 |
| Phosphate | 2 | 0.26 |
| Nitrite | 0.1 | 0.01 |
| Ascorbate | 0.4 | 0.05 |
| Sugar | 5 | 0.65 |
| Sodium glutamate | 1 | 0.13 |
| Water | 58.3 | |
| Total | 100 | |

### EXAMPLE 2

### Bacon Containing HCA's

Besides loading 15 kg of a pickling solution of the recipe shown in Table 1 into 100 kg of pork, which had been removed of bones and extraneous matter, adjusted in the amount and thickness of fat tissue, and cut to portions of suitable size, to prepare pickled meat, a processed meat food by this invention (bacon containing HCA's) was produced through conventional processes.

### EXAMPLE 3

### Sausage Containing HCA's

Besides pickling and mixing red pork meat, which had been removed of bones and extraneous matter, adjusted in the amount and thickness of fat tissue, cut to portions of suitable size, and chopped, with other blended components in accordance to the blending recipe of Table 3 in a mixer, a processed meat food (sausage containing HCA's (containing 1.5 weight % of HCA)) was produced through conventional processes.

TABLE 3

| | Blended |
|---|---|
| | composition % |
| Garcinia Powder J | 2.5 |
| Red pork meat | 66 |
| Pork fat | 21 |
| Water | 7.5 |
| Common salt | 1.5 |
| Phosphate | 0.2 |
| Nitrite | 0.01 |
| Ascorbate | 0.04 |
| Sugar | 0.5 |
| Spices (white | 0.45 |
| pepper, onion, | |
| garlic) | |
| Sodium glutamate | 0.3 |
| Total | 100 |

### EXAMPLE 4

### Hamburger Containing HCA's

Besides mixing red beef, which had been removed of bones and extraneous matter, adjusted in the amount and thickness of fat tissue, cut to portions of suitable size, and chopped, with other blended components in accordance to the blending recipe of Table 4 in a mixer, a processed meat food (hamburger containing HCA's (containing 1.5 weight % of HCA)) was produced through conventional processes.

TABLE 4

| | Blended |
|---|---|
| | composition % |
| Garcinia Powder J | 2.5 |
| Red beef | 68 |
| Beef fat | 12 |
| Water | 15.5 |
| Common salt | 1.6 |
| Spices (white | 0.3 |
| pepper, onion, | |
| garlic) | |
| Sodium glutamate | 0.1 |
| Total | 100 |

### EXAMPLE 5

### Meatballs Containing HCA's

Besides mixing chicken meat, which had been removed of bones and extraneous matter, adjusted in the amount and thickness of fat tissue, cut to portions of suitable size, and chopped, with other blended components in accordance to the blending recipe of Table 5 in a mixer, a processed meat food (meat balls containing HCA's (containing 1.5 weight % of HCA)) was produced through conventional processes.

TABLE 5

| | Blended |
|---|---|
| | composition % |
| Garcinia Powder J | 2.5 |
| Chicken meat | 85 |
| Onions | 5 |
| Water | 1.5 |
| Common salt | 2 |
| Spices (white | 0.5 |
| pepper, onion, | |
| garlic) | |
| Sodium glutamate | 0.5 |
| Starch | 3 |
| Total | 100 |

### TEST EXAMPLE 1

### Physical Endurance Fortification Effect

(1) Subjects

Six male soccer players of T University of the Republic of Korea were chosen as subjects. The physical characteristics of the subjects were as shown in Table 6.

TABLE 6

| | | Height | Weight | Body fat | VO₂max |
|---|---|---|---|---|---|
| | Age | (cm) | (kg) | (%) | (ml/min/kg) |
| A | 20 | 178.50 | 78.60 | 10.50 | 58.73 |
| B | 19 | 177.60 | 70.30 | 10.60 | 61.42 |
| C | 20 | 185.20 | 70.00 | 9.70 | 64.47 |
| D | 20 | 173.50 | 66.90 | 11.90 | 63.48 |
| E | 19 | 178.00 | 75.50 | 10.50 | 56.37 |
| F | 18 | 174.00 | 76.00 | 10.60 | 54.99 |
| mean ± | 19.33 ± | 177.80 ± | 72.88 ± | 10.63 ± | 59.91 ± |
| SD | 0.82 | 3.70 | 3.85 | 0.91 | 3.85 |

(2) Experimental Method

\mathring{1} Measurement of Maximum Oxygen Intake

Maximum oxygen intakes were measured using the method of ACSM (American College Sports Medicine, 1991). To be more specific, the subjects were made to perform two minutes of the warming-up exercise of pedaling a bicycle ergometer (REHCOR, Schiller, USA) at 50 rpm, take two minutes of rest, and then start exercise with an initial load of 0.5 KP (25 watts) and the load being increased by 0. 5 KP at every two-minute intervals. In this process, the gas ventilation volume was measured and the exhaled gas was measured and analyzed continuously using an oxygen/carbon dioxide analyzer (QUARK PFT, COSMED, Italy) to determine the maximum oxygen intake.

\mathring{2} Measurement of Exercise Sustaining Duration and Body Fat Combustion Rate

The test was performed at one week intervals for a control (ordinary ham that does not contains HCA's), pills containing 250 mg of HCA and ordinary ham, a ham containing 250 mg of HCA, pills containing 750 mg of HCA and ordinary ham, and a ham containing 750 mg of HCA, in that order. In each test, a subject was made to take in a prescribed diet (640 kcal) and a sample once a day at the time of breakfast for four days, and on the fifth day, on which measurements were made, the subject was made to perform exercise upon taking a rest for two hours after taking the prescribed diet (640 kcal) and the sample. Prior to exercise, a three-way catheter (21G) was inserted into the brachial vein for injection of physiological saline solution (1 ml/min) during exercise. The exercise load was set at 60% of the maximum oxygen intake for the first 60 minutes, and thereafter exercise was continued at a load of 80% until exhaustion. During exercise, the exhaled gas was measured and analyzed continuously using an oxygen carbon dioxide analyzer (QUARK PFT, COSMED, Italy) to determine the body fat combustion rate. All measurement results were expressed as mean±mean error. Second-order iterated variance analysis was carried out for the test among the respective groups, and the exercise sustaining duration was determined by first-order iterated variance analysis. The multiple comparison test was performed at a level of p<0.05 by Fisher PLSD.

The results concerning the fortification of physical endurance are shown in FIG. 1 (results for 250 mg of HCA) and FIG. 2 (results for 750 mg of HCA).

Both of these Figures show that in comparison to the case where HCA is taken in the form of pills, the exercise duration is significantly and clearly sustained in the case where ham with HCA mixed in is taken.

The results concerning the promotion of body fat combustion are shown in FIG. 3. FIG. 3 (results for 250 mg of HCA) clearly shows that in comparison to the control and other cases, the oxidation rate, which expresses the body fat combustion rate, is increased in the case where ham with HCA is mixed in is taken.

### BRIEF DESCRIPTION OF THE DRAWINGS

FIG. 1 shows the exercise sustaining duration in the case of intake of 250 mg of HCA. The ordinate axis indicates the time (minutes) until exhaustion. The leftmost column indicates the results for the group (control group) that took in ordinary ham that does not contain HCA, the central column indicates the results for the group that took in pills containing 250 mg of HCA at the same time as ordinary ham, and the rightmost column indicates the results for the group that took in ham containing 250 mg of HCA. In the Figure, the symbols a and b indicate the multiple comparison test results and the results concerning different symbols indicate that there is a significant difference within a risk factor of 5% or less.

FIG. 2 shows the exercise sustaining duration in the case of intake of 750 mg of HCA. The ordinate axis indicates the time (minutes) until exhaustion. The leftmost column indicates the results for the group (control group) that took in ordinary ham that does not contain HCA, the central column indicates the results for the group that took in pills containing 750 mg of HCA at the same time as ordinary ham, and the rightmost column indicates the results for the group that took in ham containing 750 mg of HCA. In the Figure, the symbols a, b, and ab indicate the multiple comparison test results and the results concerning different symbols indicate that there is a significant difference within a risk factor of 5% or less.

FIG. 3 shows the transition of the body fat oxidation rate in the case of intake of 250 mg of HCA. The ordinate axis indicates the body fat oxidation rate (mg/kg/5 minutes) and the abscissa axis indicates the exercise time (minutes). -◯- indicates the transition for the group that took in ham containing 250 mg of HCA, -●- indicates the transition for the group that took in pills containing 250 mg of HCA, and -□- indicates the transition for the group (control group) that took in ordinary ham that does not contain HCA.

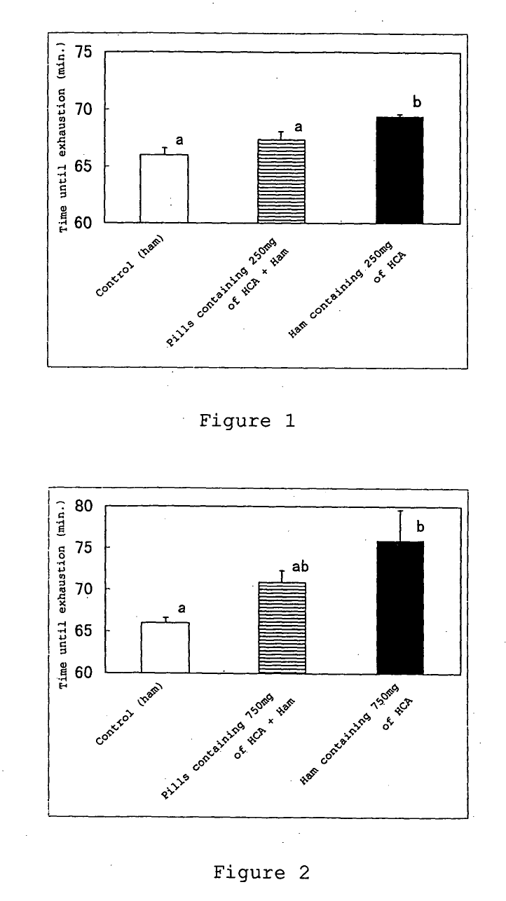

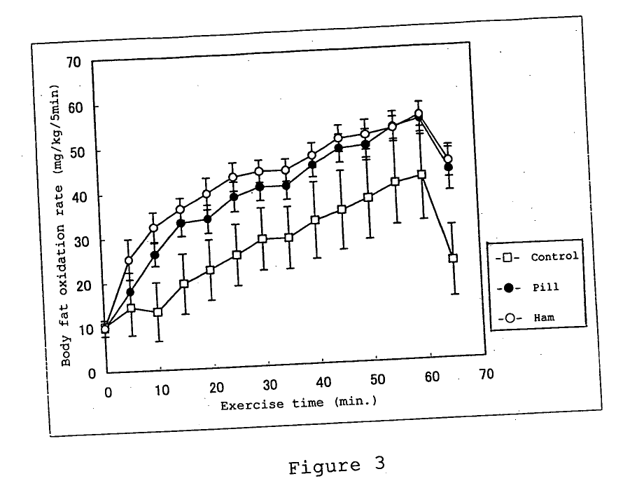

1. A processed meat food, having (−)-hydroxycitric acid or its lactone form or a salt of either (−)-hydroxycitric acid or its lactone form (these shall be referred to hereinafter in the claims as “HCA's”) mixed therein.

2. A processed meat food, having mixed therein a Garcinia rind extract, which contains HCA's.

3. A processed meat food as set forth in claim 2, wherein the Garcinia is goraka (Garcinia cambogia Desr.), Indian mangosteen (Garcinia indica Choisy), or Asam gelugor (Garcinia atroviridis Griff.).

4. A processed meat food as set forth in any one of claims 1 through 3, wherein the processed meat food is a meat semi-product, meat product, or meat-containing dish (including semi-products).

5. A processed meat food as set forth in any one of claims 1 through 3, wherein the processed meat food is selected from among the group comprised of hamburgers, Vienna sausages, raw seasoned meats, raw roast beefs, cooked seasoned meats, pork cutlets, meat hams, meat sausages, meat bacons, meat corned beefs, roasted meats, dried meats, fish meat hamburgers, meat balls (including fish meat balls), minced meat cutlets, nuggets, chicken cutlets, beef cutlets, shao-mai, jiao-zi, croquettes, spring rolls, kamaboko (boiled fish paste), chikuwa (fish paste), rolled omelets, tsumire (dumplings for soup), hanpen (fish cake), and satsuma-age (fried fish balls).

6. A processed meat food as set forth in any one of claims 1 through 3, wherein HCA's are contained at an amount in the range of 1 mg to 30 g per 100 g of processed meat food.

7. The use of HCA's for the production of processed meat foods for physical endurance fortification, which have HCA's mixed therein.

8. The use of Garcinia rind extract for the production of processed meat foods for physical endurance fortification, which have mixed therein Garcinia rind extract containing HCA's.

9. The use of Garcinia rind extract as set forth in claim 8, wherein the Garcinia is goraka (Garcinia cambogia Desr.), Indian mangosteen (Garcinia indica Choisy), or Asam gelugor (Garcinia atroviridis Griff.).

10. The use of HCA's as set forth in claim 7, wherein the processed meat food is a meat semi-product, meat product, or meat-containing dish (including semi-products).

11. The use of Garcinia rind extract as set forth in claim 8 or 9, wherein the processed meat food is a meat semi-product, meat product, or meat-containing dish (including semi-products).

12. The use of HCA's as set forth in claim 7, wherein the processed meat food is selected from among the group comprised of hamburgers, Vienna sausages, raw seasoned meats, raw roast beefs, cooked seasoned meats, pork cutlets, meat hams, meat sausages, meat bacons, meat corned beefs, roasted meats, dried meats, fish meat hamburgers, meat balls (including fish meat balls), minced meat cutlets, nuggets, chicken cutlets, beef cutlets, shao-mai, jiao-zi, croquettes, spring rolls, kamaboko (boiled fish paste), chikuwa (fish paste), rolled omelets, tsumire (dumplings for soup), hanpen (fish cake), and satsuma-age (fried fish balls).

13. The use of Garcinia rind extract as set forth in claim 8 or 9, wherein the processed meat food is selected from among the group comprised of hamburgers, Vienna sausages, raw seasoned meats, raw roast beefs, cooked seasoned meats, pork cutlets, meat hams, meat sausages, meat bacons, meat corned beefs, roasted meats, dried meats, fish meat hamburgers, meat balls (including fish meat balls), minced meat cutlets, nuggets, chicken cutlets, beef cutlets, shao-mai, jiao-zi, croquettes, spring rolls, kamaboko (boiled fish paste), chikuwa (fish paste), rolled omelets, tsumire (dumplings for soup), hanpen (fish cake), and satsuma-age (fried fish balls).

14. The use of HCA's as set forth in claim 7, wherein the processed meat food for physical endurance fortification contains HCA's at an amount in the range of 1 mg to 30 g per 100 g.

15. The use of Garcinia rind extract as set forth in claim 8 or 9, wherein the processed meat food for physical endurance fortification contains HCA's at an amount in the range of 1 mg to 30 g per 100 g.

16. A method of fortifying the physical endurance of a human being or other animal, wherein a processed meat food, having HCA's mixed therein, is taken in by the human being or other animal.

17. A method of fortifying the physical endurance of a human being or other animal, wherein a processed meat food, having mixed therein a Garcinia rind extract containing HCA's, is taken in by the human being or other animal.

18. A method of fortifying the physical endurance of a human being or other animal as set forth in claim 17, wherein the Garcinia is goraka (Garcinia cambogia Desr.), Indian mangosteen (Garcinia indica Choisy), or Asam gelugor (Garcinia atroviridis Griff.).

19. A method of fortifying the physical endurance of a human being or other animal as set forth in any one of claims 16 through 18, wherein the processed meat food is a meat semi-product, meat product, or meat-containing dish (including semi-products).

20. A method of fortifying the physical endurance of a human being or other animal as set forth in any one of claims 16 through 18, wherein the processed meat food is selected from among the group comprised of hamburgers, Vienna sausages, raw seasoned meats, raw roast beefs, cooked seasoned meats, pork cutlets, meat hams, meat sausages, meat bacons, meat corned beefs, roasted meats, dried meats, fish meat hamburgers, meat balls (including fish meat balls), minced meat cutlets, nuggets, chicken cutlets, beef cutlets, shao-mai, jiao-zi, croquettes, spring rolls, kamaboko (boiled fish paste), chikuwa (fish paste), rolled omelets, tsumire (dumplings for soup), hanpen (fish cake), and satsuma-age (fried fish balls).

21. A method of fortifying the physical endurance of a human being or other animal as set forth in any one of claims 16 through 18, wherein the processed meat food contains HCA's at an amount in the range of 1 mg to 30 g per 100 g.

22. The use of HCA's for the production of processed meat foods for body fat combustion promotion, which have HCA's mixed therein.

23. The use of Garcinia rind extract for the production of processed meat foods for body fat combustion promotion, which have mixed therein Garcinia rind extract containing HCA's.

24. The use of Garcinia rind extract as set forth in claim 23, wherein the Garcinia is goraka (Garcinia cambogia Desr.), Indian mangosteen (Garcinia indica Choisy), or Asam gelugor (Garcinia atroviridis Griff.).

25. The use of HCA's as set forth in claim 22, wherein the processed meat food is a meat semi-product, meat product, or meat-containing dish (including semi-products).

26. The use of Garcinia rind extract as set forth in claim 23 or 24, wherein the processed meat food is a meat semi-product, meat product, or meat-containing dish (including semi-products).

27. The use of HCA's as set forth in claim 22, wherein the processed meat food is selected from among the group comprised of hamburgers, Vienna sausages, raw seasoned meats, raw roast beefs, cooked seasoned meats, pork cutlets, meat hams, meat sausages, meat bacons, meat corned beefs, roasted meats, dried meats, fish meat hamburgers, meat balls (including fish meat balls), minced meat cutlets, nuggets, chicken cutlets, beef cutlets, shao-mai, jiao-zi, croquettes, spring rolls, kamaboko (boiled fish paste), chikuwa (fish paste), rolled omelets, tsumire (dumplings for soup), hanpen (fish cake), and satsuma-age (fried fish balls).

28. The use of Garcinia rind extract as set forth in claim 23 or 24, wherein the processed meat food is selected from among the group comprised of hamburgers, Vienna sausages, raw seasoned meats, raw roast beefs, cooked seasoned meats, pork cutlets, meat hams, meat sausages, meat bacons, meat corned beefs, roasted meats, dried meats, fish meat hamburgers, meat balls (including fish meat balls), minced meat cutlets, nuggets, chicken cutlets, beef cutlets, shao-mai, jiao-zi, croquettes, spring rolls, kamaboko (boiled fish paste), chikuwa (fish paste), rolled omelets, tsumire (dumplings for soup), hanpen (fish cake), and satsuma-age (fried fish balls).

29. The use of HCA's as set forth in claim 22, wherein the processed meat food for body fat combustion promotion contains HCA's at an amount in the range of 1 mg to 30 g per 100 g.

30. The use of Garcinia rind extract as set forth in claim 23 or 24, wherein the processed meat food for body fat combustion promotion contains HCA's at an amount in the range of 1 mg to 30 g per 10 g.

31. A method of promoting body fat combustion by a human being or other animal, wherein a processed meat food, having HCA's mixed therein, is taken in by the-human being or other animal.

32. A method of promoting body fat combustion by a human being or other animal, wherein a processed meat food, having mixed therein a Garcinia rind extract containing HCA's, is taken in by the human being or other animal.

33. A method of promoting body fat combustion by a human being or other animal as set forth in claim 32, wherein the Garcinia is goraka (Garcinia cambogia Desr.), Indian mangosteen (Garcinia indica Choisy), or Asam gelugor (Garcinia atroviridis Griff.).

34. A method of promoting body fat combustion by a human being or other animal as set forth in any one of claims 31 through 33, wherein the processed meat food is a meat semi-product, meat product, or meat-containing dish (including semi-products).

35. A method of promoting body fat combustion by a human being or other animal as set forth in any one of claims 31 through 33, wherein the processed meat food is selected from among the group comprised of hamburgers, Vienna sausages, raw seasoned meats, raw roast beefs, cooked seasoned meats, pork cutlets, meat hams, meat sausages, meat bacons, meat corned beefs, roasted meats, dried meats, fish meat hamburgers, meat balls (including fish meat balls), minced meat cutlets, nuggets, chicken cutlets, beef cutlets, shao-mai, jiao-zi, croquettes, spring rolls, kamaboko (boiled fish paste), chikuwa (fish paste), rolled omelets, tsumire (dumplings for soup), hanpen (fish cake), and satsuma-age (fried fish balls).

36. A method of promoting body fat combustion by a human being or other animal as set forth in any one of claims 31 through 33, wherein the processed meat food contains HCA's at an amount in the range of 1 mg to 30 g per 100 g.
